(19) United States
(12) Patent Application Publication
(10) Pub. No.: US 2026/0014369 A1
(43) Pub. Date: Jan. 15, 2026

(54) Self-Curling Cochlear Electrode Lead and Method of Manufacturing the Same

(71) Applicant: Advanced Bionics LLC, Valencia, CA (US)

(72) Inventors: Nicholas V. Apollo, Valencia, CA (US); Anil K. Patnala, Stevenson Ranch, CA (US); Kanthaiah Koka, Valencia, CA (US); Anthony J. Spahr, Newhall, CA (US); Candelaria Salvatierra, West Lancaster, CA (US); Mark B. Downing, Valencia, CA (US); Volkmar Hamacher, Hannover (DE)

(21) Appl. No.: 19/264,261

(22) Filed: Jul. 9, 2025

Related U.S. Application Data
(60) Provisional application No. 63/669,513, filed on Jul. 10, 2024.

(51) Int. Cl. A61N 1/05

(52) U.S. Cl. CPC A61N 1/0541

(57) ABSTRACT

An exemplary self-curling cochlear electrode lead adapted for insertion into a human cochlea, comprises: a flexible body formed of a flexible insulating material; a shape memory polymer element that is embedded within the flexible body and that is configured to cause the self-curling cochlear electrode lead to transition to a curved spiral shape so as to conform with a curvature of the human cochlea in response the shape memory polymer element being subjected to a transition agent. The flexible body may include a plurality of transition agent vias configured to transmit the transition agent through the flexible body to the shape memory polymer element.

### RELATED APPLICATIONS

The present application claims priority to U.S. Provisional Patent Application No. 63/669,513, filed Jul. 10, 2024, the contents of which is hereby incorporated by reference in its entirety.

### BACKGROUND INFORMATION

Cochlear implant systems are used to provide, restore, and/or improve hearing loss suffered by cochlear implant patients who use the cochlear implant systems. A key component of a cochlear implant system is an electrode lead that is inserted into a cochlea of the patient in a delicate surgical procedure referred to herein as an “insertion procedure.” Insertion procedures are difficult due to the structure of the human cochlea, which is in the shape of a spiral beginning at a base and ending at an apex. If the electrode lead is not positioned correctly, cochlear trauma and/or an inferior hearing outcome for the patient may occur.

Current cochlear electrode lead technologies include two general designs: straight cochlear electrode leads and pre-curved cochlear electrode leads. The insertion procedure for straight cochlear electrode leads includes the straight cochlear electrode lead generally following a trajectory of a lateral wall of the scala tympani. Unfortunately, straight cochlear electrode leads have a drawback in that they typically reside far away from the modiolus of the cochlea when inserted, which results in lower specificity in neural activation and a potentially inferior hearing outcome for the patient.

Pre-curved cochlear electrode leads are manufactured in an already-curled shape and are straightened before implantation using either a stylet that is inserted into a lumen of the pre-curved cochlear electrode lead or by using a straight rigid sheath provided around the pre-curved cochlear electrode lead. While a surgeon inserts a pre-curved cochlear electrode lead into the cochlea, the stylet or sheath is gradually withdrawn, which allows the pre-curved cochlear electrode lead to return to its curled shape and conform with the helical shape of the cochlea. Typically, specialized surgical tools and surgical techniques are required to handle the pre-curved cochlear electrode and remove the stylet or sheath. Such techniques can be challenging and require specialized training and experience to perform correctly. Improper insertion of a pre-curved cochlear electrode lead can result in damage to the electrode lead, damage to the cochlear tissue, and/or improper electrode placement in the cochlea (e.g., translocation, tip foldover, etc.). Moreover, typical pre-curved cochlear electrode leads tend to only reach a moderate insertion depth into the cochlea. This results in limited access to more apical spiral ganglion cells that encode low-frequency sounds, a vital component of speech understanding and music appreciation.

Some have proposed using a shape memory alloy such as nitinol to cause a cochlear electrode lead to self-curl upon reaching a transition temperature. However, even though the composition of nitinol may be adjusted to achieve a modulus transition near body temperature, the rate of modulus change cannot be decreased to a useful, optimized rate. This results in an electrode lead that curls too quickly and/or forcefully upon insertion into the cochlea and requires the surgeon to match the rate of insertion to the rate of modulus change for the insertion to be successful. If such an electrode lead is inserted too slowly relative to the change in curve of the electrode lead, a tip foldover or a scalar translocation may occur. In addition, the phase change of nitinol is influenced only by temperature. As such, nitinol-based self-curling cochlear electrode leads may be prone to an increased risk of premature curling (e.g., when positioned under hot operating room lights). For at least these reasons, nitinol-based self-curling cochlear electrode leads are unrealistic.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary cochlear implant system according to principles described herein.

FIG. 2 illustrates a schematic structure of the human cochlea according to principles described herein.

FIGS. 3A-3C illustrate an exemplary insertion procedure of a self-curling cochlear electrode lead according to principles described herein.

FIGS. 4-13 show exemplary self-curling cochlear electrode leads that may be implemented according to principles described herein.

FIG. 14 shows different exemplary longitudinal shapes of shape memory polymer elements according to principles described herein.

FIG. 15 shows a diagram depicting exemplary designs of shape memory polymer elements that may be laser cut from a single sheet of shape memory polymer material according to principles described herein.

FIG. 16 shows an exemplary method for manufacturing a self-curling cochlear electrode lead according to principles described herein.

### DETAILED DESCRIPTION

A self-curling cochlear electrode lead and methods for manufacturing the same are described herein. To overcome the aforementioned problems, the self-curling cochlear electrode leads described herein are configured to both self-curl to a mid-scalar or perimodiolar position and also extend more apically into the cochlea after insertion to gain access to low-frequency spiral ganglion cells. The exemplary self-curling cochlear electrode leads described herein achieve these goals by employing designs that have optimal self-curling properties and/or advantageous methods of curling.

As will be described in more detail below, the exemplary self-curling cochlear electrode leads described herein may include a plurality of electrode contacts, a plurality of wires connected to the plurality of electrode contacts, and a portion that includes a shape memory polymer that allows the cochlear electrode lead to transition (e.g., from a substantially straightened position) to a curved spiral shape so as to conform with a curvature of a human cochlea. As used herein, a “shape memory polymer” refers to a type of polymeric material that has the ability to transition from a temporary shape (e.g., a substantially straight shape) to a permanent shape in response to being subjected to a transition agent. As used herein, the expression “shape memory” may correspond to any transition in shape that may occur in a material such as a polymer as a result of the material being exposed to a transition agent. As used herein, a “transition agent” may correspond to any type of agent that may be used to cause or otherwise facilitate a shape memory polymer element changing shape. For example, a transition agent may include moisture in the form of water or bodily fluid within a recipient of a self-curling cochlear electrode lead. Additionally or alternatively, a transition agent may include heat applied to the self-curling cochlear electrode lead at least one of during insertion or after insertion. In examples where the transition agent includes heat, the heat may be applied in any suitable manner. For example, the heat may be applied to the self-curling cochlear electrode lead by at least one of body temperature of a recipient of the self-curling cochlear electrode lead, an external heat source (e.g., an IR laser), or heat generated by way of the plurality of electrode contacts.

As will be described further herein, the flexible body of a self-curling cochlear electrode lead may include a plurality of transition agent vias configured to transmit the transition agent through the flexible body to the shape memory polymer element. Examples of transition agent vias and how the transition agent vias may facilitate a self-curling cochlear electrode lead to transition to a curved spiral shape are described herein.

The self-curling cochlear electrode leads described herein may provide various benefits to recipients of cochlear implants, as well as to surgeons and others involved with insertion procedures. For example, because the cochlear electrode leads described herein may come straight and self-curl after insertion, a surgeon is able to insert the self-curling cochlear electrode lead in a manner that does not require a specialized insertion tool and/or an advanced insertion technique. In addition, self-curling cochlear electrode leads such as those described herein are configured to be stiff enough to maintain a substantially straight configuration before being inserted into the cochlea, but compliant enough while in the straight configuration to flex to some degree when inserted into the cochlea in order to minimize or prevent damage to the cochlea as the self-curling cochlear electrode leads come into contact with walls and/or other structures of the cochlea. Further, self-curling cochlear electrode leads such as those described herein may beneficially travel along the lateral wall of the scala tympani upon initial insertion into the cochlea, move toward the modiolus at a pre-defined rate after insertion into the cochlea, extend more toward the apex of the cochlea than conventional pre-curved cochlear electrode leads, and employ optimal self-curling characteristics that ensure the cochlear electrode lead progresses in a desirable trajectory. In addition, self-curling cochlear electrode leads such as those described herein are more easily compatible with robotic surgery as compared to conventional cochlear electrode leads. Moreover, in certain examples, the self-curling cochlear electrode leads described herein may not include a lumen that is configured to receive a stylet. Because of this, it may be possible to make self-curling cochlear electrode leads such as those described herein thinner and more compliant than conventional pre-curved cochlear electrode leads, which increases the likelihood of having an atraumatic insertion into the cochlea.

In addition, the methods of manufacturing a self-curling cochlear electrode lead described herein are beneficial in that they simplify the manufacturing process and/or reduce manufacturing costs as compared to manufacturing conventional pre-curved cochlear electrode leads. Conventional pre-curved cochlear electrode leads are typically formed using a cochlear electrode lead mold that includes a portion designed to form a lumen that has a specific shape and dimensions. Changing the shape and dimensions of the lumen in a conventional pre-curved cochlear electrode lead typically requires manufacturing a new cochlear electrode lead mold, which is costly and time consuming. In contrast, because the self-curling cochlear electrode leads described herein may not position the shape memory polymer within a lumen, there is no need to design the shape memory polymer to fit within a specific lumen. In addition, with the self-curling cochlear electrode leads described herein, the design of the shape memory polymer can be changed without having to manufacture a new cochlear electrode lead mold, which results in reduced manufacturing costs. Further, the methods of manufacturing a self-curling cochlear electrode lead described herein may not require multiple polymer components to be assembled or adhered together, which results in a simplified manufacturing process.

Various embodiments will now be described in more detail with reference to the figures. The disclosed apparatus and methods may provide one or more of the benefits mentioned above and/or various additional and/or alternative benefits that will be made apparent herein.

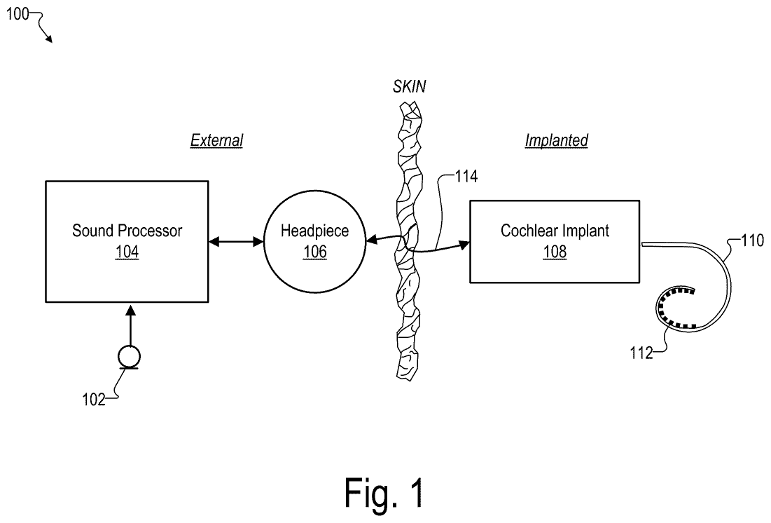

FIG. 1 illustrates an exemplary cochlear implant system 100. As shown, cochlear implant system 100 may include a microphone 102, a sound processor 104, a headpiece 106 having a coil disposed therein, a cochlear implant 108, and a self-curling cochlear electrode lead 110 (“electrode lead 110”). Electrode lead 110 may include an array of electrodes 112 disposed on a distal portion of electrode lead 110 and that are configured to be inserted into the cochlea to stimulate the cochlea after the distal portion of electrode lead 110 is inserted into the cochlea. It will be understood that one or more other electrodes (e.g., including a ground electrode, not explicitly shown) may also be disposed on other parts of electrode lead 110 (e.g., on a proximal portion of electrode lead 110) to, for example, provide a current return path for stimulation current generated by electrodes 112 and to remain external to the cochlea after electrode lead 110 is inserted into the cochlea. Various embodiments of self-curling cochlear electrode lead 110 will be described herein. Additional or alternative components may be included within cochlear implant system 100 as may serve a particular implementation. For example, a pre-curved electrode lead and/or a straight electrode lead may alternatively be used in connection with cochlear implant 108.

As shown, cochlear implant system 100 may include various components configured to be located external to a patient including, but not limited to, microphone 102, sound processor 104, and headpiece 106. Cochlear implant system 100 may further include various components configured to be implanted within the patient including, but not limited to, cochlear implant 108 and electrode lead 110.

Microphone 102 may be configured to detect audio signals presented to the user. Microphone 102 may be implemented in any suitable manner. For example, microphone 102 may include a microphone that is configured to be placed within the concha of the ear near the entrance to the ear canal, such as a T-MIC™ microphone from Advanced Bionics. Such a microphone may be held within the concha of the ear near the entrance of the ear canal by a boom or stalk that is attached to an ear hook configured to be selectively attached to sound processor 104. Additionally or alternatively, microphone 102 may be implemented by one or more microphones disposed within headpiece 106, one or more microphones disposed within sound processor 104, one or more beam-forming microphones, and/or any other suitable microphone as may serve a particular implementation.

Sound processor 104 (i.e., one or more components included within sound processor 104) may be configured to direct cochlear implant 108 to generate and apply electrical stimulation (also referred to herein as “stimulation current”) representative of one or more audio signals (e.g., one or more audio signals detected by microphone 102, input by way of an auxiliary audio input port, input by way of a device like the Clinical Programming Interface (“CPI”) device from Advanced Bionics, etc.) to one or more stimulation sites associated with an auditory pathway (e.g., the auditory nerve) of the patient. Exemplary stimulation sites include, but are not limited to, one or more locations within the cochlea, the cochlear nucleus, the inferior colliculus, and/or any other nuclei in the auditory pathway. To this end, sound processor 104 may process the one or more audio signals in accordance with a selected sound processing strategy or program to generate appropriate stimulation parameters for controlling cochlear implant 108. Sound processor 104 may be housed within any suitable housing (e.g., a behind-the-ear (“BTE”) unit, a body worn device, headpiece 106, and/or any other sound processing unit as may serve a particular implementation).

In some examples, sound processor 104 may wirelessly transmit stimulation parameters (e.g., in the form of data words included in a forward telemetry sequence) and/or power signals to cochlear implant 108 by way of a wireless communication link 114 between headpiece 106 and cochlear implant 108 (e.g., a wireless link between a coil disposed within headpiece 106 and a coil physically coupled to cochlear implant 108). It will be understood that communication link 114 may include a bi-directional communication link and/or one or more dedicated uni-directional communication links.

Headpiece 106 may be communicatively coupled to sound processor 104 and may include an external antenna (e.g., a coil and/or one or more wireless communication components) configured to facilitate selective wireless coupling of sound processor 104 to cochlear implant 108. Headpiece 106 may additionally or alternatively be used to selectively and wirelessly couple any other external device to cochlear implant 108. To this end, headpiece 106 may be configured to be affixed to the patient's head and positioned such that the external antenna housed within headpiece 106 is communicatively coupled to a corresponding implantable antenna (which may also be implemented by a coil and/or one or more wireless communication components) included within or otherwise associated with cochlear implant 108. In this manner, stimulation parameters and/or power signals may be wirelessly transmitted between sound processor 104 and cochlear implant 108 via a communication link 114 (which may include a bi-directional communication link and/or one or more dedicated uni-directional communication links as may serve a particular implementation).

Cochlear implant 108 may include any type of implantable stimulator that may be used in association with the systems and methods described herein. For example, cochlear implant 108 may be implemented by an implantable cochlear stimulator. In some alternative implementations, cochlear implant 108 may include a brainstem implant and/or any other type of cochlear implant that may be implanted within a patient and configured to apply stimulation to one or more stimulation sites located along an auditory pathway of a patient.

In some examples, cochlear implant 108 may be configured to generate electrical stimulation representative of an audio signal processed by sound processor 104 (e.g., an audio signal detected by microphone 102) in accordance with one or more stimulation parameters transmitted thereto by sound processor 104. Cochlear implant 108 may be further configured to apply the electrical stimulation to one or more stimulation sites (e.g., one or more intracochlear regions) within the patient via electrodes 112 disposed along electrode lead 110. In some examples, cochlear implant 108 may include a plurality of independent current sources each associated with a channel defined by one or more of electrodes 112. In this manner, different stimulation current levels may be applied to multiple stimulation sites simultaneously by way of multiple electrodes 112.

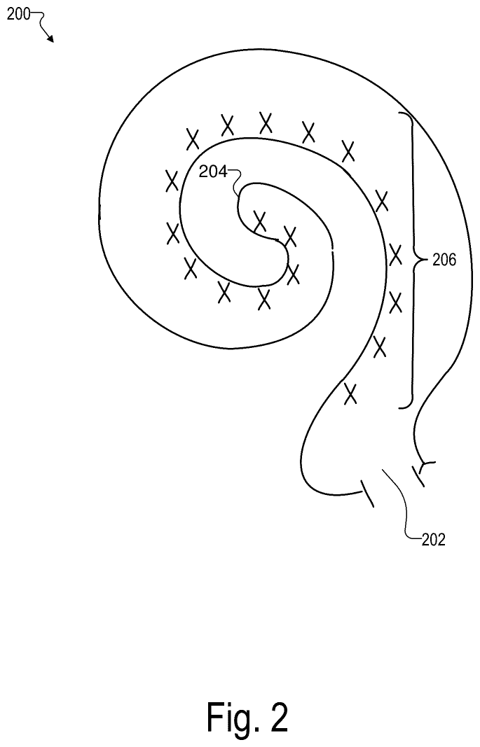

FIG. 2 illustrates a schematic structure of the human cochlea 200 into which electrode lead 110 may be inserted. As shown in FIG. 2, cochlea 200 is in the shape of a spiral beginning at a base 202 and ending at an apex 204. Within cochlea 200 resides auditory nerve tissue 206, which is denoted by Xs in FIG. 2. The auditory nerve tissue 206 is organized within the cochlea 200 in a tonotopic manner. Relatively low frequencies are encoded at or near the apex 204 of the cochlea 200 (referred to as an “apical region”) while relatively high frequencies are encoded at or near the base 202 (referred to as a “basal region”). Hence, electrical stimulation applied by way of electrodes disposed within the apical region (i.e., “apical electrodes”) may result in the patient perceiving relatively low frequencies and electrical stimulation applied by way of electrodes disposed within the basal region (i.e., “basal electrodes”) may result in the patient perceiving relatively high frequencies. The delineation between the apical and basal electrodes on a particular electrode lead may vary depending on the insertion depth of the electrode lead, the anatomy of the patient's cochlea, and/or any other factor as may serve a particular implementation.

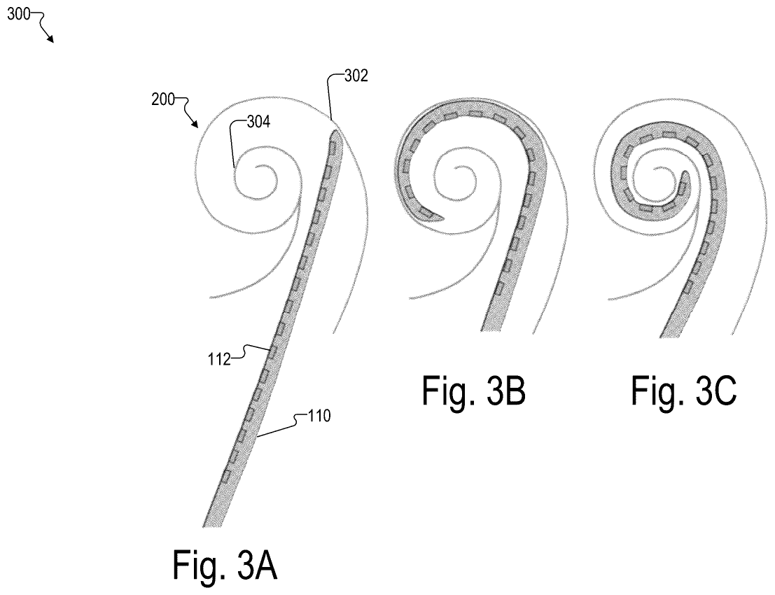

FIGS. 3A-3C illustrate an exemplary insertion procedure 300 that shows how self-curling cochlear electrode lead 110 may transition from a straight configuration to a curved configuration upon insertion in cochlea 200. Prior to self-curling cochlear electrode lead 110 being inserted into the patient's cochlea, self-curling cochlear electrode lead 110 may be provided in a straight configuration. For example, self-curling cochlear electrode lead 110 may be heated, bent into the straight configuration, and allowed to cool while being held in the straight configuration. Self-curling cochlear electrode lead 110 may be configured to maintain the straight configuration at room temperature (e.g., approximately 23° C.) but transition to the curved configuration once self-curling cochlear electrode lead 110 is exposed to a transition agent (e.g., bodily fluid and/or heat).

As illustrated in FIG. 3A, insertion procedure 300 may involve inserting self-curling cochlear electrode lead 110 while in the straight configuration through an entry point (e.g., within a round window or cochleostomy of cochlea 200, or another suitable location) and into a scala tympani of cochlea 200. Self-curling cochlear electrode lead 110 may be sufficiently compliant that, as the insertion of self-curling cochlear electrode lead 110 proceeds, self-curling cochlear electrode lead 110 may follow, as shown in FIG. 3B, a lateral wall 302 of the scala tympani without causing damage to cochlea 200. Once self-curling cochlear electrode lead 110 is exposed to a transition agent, the shape memory polymer causes, facilitates, and/or allows self-curling cochlear electrode lead 110 to transition to the curved configuration in which self-curling cochlear electrode lead 110 has a curved spiral shape that conforms with a curvature of cochlea 200. As shown in FIG. 3C, the change in transition agent causes self-curling cochlear electrode lead 110 to self-curl away from lateral wall 302 and toward the modiolus 304 of cochlea 200. In so doing, self-curling cochlear electrode lead 110 is also able to extend more toward the apex of cochlea 200 than traditional pre-curved cochlear electrode leads.

The shape memory polymer used in self-curling cochlear electrode lead 110 may include any suitable polymer that is configured to transition from a temporary shape (e.g., a straightened configuration) to its permanent shape once the shape memory polymer is exposed to a transition agent. Examples of shape memory polymers that may be utilized in self-curling cochlear electrode leads such as those described herein may include, for example, polyurethanes, polynorbornene, poly(ε-caprolactone) combined with poly(hydroxybutyrate-co-hydroxyvalerate), and/or any other suitable shape memory polymer or combination of shape memory polymers. In certain examples, a shape memory polymer element may be permanent or transient. For example, a shape memory polymer element may be biodegradable or water soluble in certain implementations. In certain alternative examples, a shape memory polymer element may be non-biodegradable and may be configured to remain during the operational life of the electrode lead.

In certain examples, a shape memory polymer element may result from a copolymerization of silicone and one or more of the exemplary materials described herein. In such examples, the entire electrode lead may be considered as a shape memory polymer element.

In examples where a transition agent includes applying moisture to a shape memory polymer element, the moisture may facilitate the shape memory polymer element curling more quickly and/or to a greater degree in areas where the moisture reaches the shape memory polymer element.

In examples where a transition agent includes applying heat to self-curling cochlear electrode lead 110, the heat may facilitate a shape-memory polymer element reach a transition temperature. As used herein, the “transition temperature” refers to the temperature at which the shape memory polymer transitions to its permanent shape from the temporary shape. In certain examples, the transition temperature may be at or near body temperature of a recipient. In certain examples, the transition temperature may be above the body temperature. In such examples, the transition temperature may be achieved through a heat source (e.g., an external light source, an electrode, etc.) other than the body temperature of the recipient. The transition temperature of self-curling cochlear electrode lead 110 may be any suitable temperature that is above room temperature but that is at or below a normal body temperature of the patient (e.g., approximately 37° C.).

In certain examples, the transition agent may include applying a combination of light and heat to a shape memory polymer element to facilitate a change in shape. For example, a shape memory polymer element may be provided with iron oxide (Fe₃O₄) particles that are configured to generate heat when irradiated with near infrared light due to plasmon resonance. In such examples, the iron oxide particles may be provided with the shape memory polymer element in any suitable manner. For example, the iron oxide particles may be blended into the polymer matrix of the shape memory polymer element or applied as a coating on an external surface of the shape memory polymer element. In certain examples, particles such as iron oxide particles may be provided with respect to only a portion of the shape memory polymer element where it is desirable to induce a shape change with heat. Other portions of the shape memory polymer element may not include such particles.

In certain examples, shape memory polymer elements such as those described herein may additionally or alternatively be triggered to transition from a temporary shape to a permanent shape based on one or more triggers other than moisture or temperature. For example, such triggers may include pH, light, ultrasound, and/or mechanical vibration. Such triggers may be internally triggered (e.g., with temperature) or externally triggered (e.g., with light provided to the recipient that transverses the tympanic membrane). In certain examples, a trigger may be provided a healing response of the body of a recipient of a self-curling cochlear electrode lead. For example, a reactive oxygen species that occurs during healing may be a trigger for a shape change in certain examples.

In certain examples, shape memory polymers such as those described herein may be configured to transition based on a combination of triggers. For example, a shape memory polymer element may be configured to transition a first amount based on a first trigger and transition a second amount different than the first amount based on a second trigger. The first trigger may correspond to a first type of trigger and second trigger may correspond to a second type of trigger different than the first type of trigger. To illustrate, the first trigger may correspond to a transition temperature that once achieved results in the shape memory polymer element having a first amount of curl. The second trigger may correspond to a moisture trigger in which moisture causes the shape memory polymer element to achieve a second amount of curl that is greater than the first amount of curl. In certain examples, the first trigger may be achieved operatively (e.g., during a lead insertion procedure) and the second trigger may be achieved post-operatively (e.g., after completion of the lead insertion procedure).

Self-curling cochlear electrode leads such as those described herein may provide one or more of the following benefits and/or features. For example, the length and/or curvature of shape memory polymer elements of self-curling cochlear electrode leads may be selectable/customizable depending on individual patient cochlea anatomy. Additionally or alternatively, self-curling cochlear electrode leads such as those described herein may utilize a lumen approach in certain examples to control the depth, curvature, and/or trajectory and thereby placement of the self-curling cochlear electrode lead within the cochlea. Additionally or alternatively, self-curling cochlear electrode leads such as those described herein may be configured to be inserted using an insertion technique that does not require a specialized insertion tool and/or an advanced insertion technique. Additionally or alternatively, self-curling cochlear electrode leads such as those described herein may provide mechanical characteristics that facilitate the self-curling cochlear electrode leads progressing in a desirable trajectory within the cochlea. Additionally or alternatively, self-curling cochlear electrode leads such as those described herein may achieve gradual self-curling at a pre-defined rate after insertion into the cochlea.

In certain examples, self-curling cochlear electrode leads such as those described herein may include one or more shape memory polymer elements that may both self-curl to a mid-scalar or perimodiolar position, but also extend more apically into the cochlea after insertion to gain access to low-frequency spiral ganglion cells. This may be achieved through a customizable electrode lead design that has optimal mechanical properties and/or an advantageous method of curling.

In certain examples, the design of a shape memory polymer element may vary in curvature, length, and/or cross-sectional dimensions to achieve an intended position of a self-curling cochlear electrode lead in the cochlea.

In certain examples, a self-curling cochlear electrode lead may have a pre-formed lumen along a length of the self-curling cochlear electrode lead that may accommodate one or more shape memory polymer elements. The pre-formed lumen may be configured such that it is possible to insert any one of a plurality of different shape memory polymer elements depending on insertion requirements and/or desired location, amount, etc. of curl. For example, a first shape memory polymer element having a first longitudinal shape may be inserted within the lumen. Alternatively, a second shape memory polymer element having a second longitudinal shape that is different than the first shape may be inserted within the lumen. Using a lumen approach together with such shape memory polymer elements may facilitate preferential characteristics of shape, bend, insertion depth, and/or trajectory inside the cochlea. In so doing, it may be possible to customize the self-curling cochlear electrode lead to provide several different combinations of slight variants of the self-curling cochlear electrode lead without having to build multiple lengths and curvatures of the self-curling cochlear electrode lead. For example, adjusting the length of a shape memory polymer element that may be provided within a pre-formed lumen may provide control on the insertion depth.

In certain examples, self-curling cochlear electrode leads such as those described herein may be customized for a particular recipient based on imaging of the recipient's cochlea. For example, pre-operative imaging (e.g., magnetic resonance imaging (MRI), computed tomography (CT), 3D cochlea reconstruction, etc.) may be used in certain examples. With such pre-operative imaging, an individual recipient's cochlea anatomy may be used to determine the desired angular coverage, curvature, and/or trajectory of the self-curling cochlear electrode lead. The shape memory polymer element(s) implemented in such examples may be configured in any suitable manner. For example, the shape memory polymer element(s) may be designed to meet the targets described above by manipulating the stiffness, the curvature, the dimensions (e.g., length, cross-section size/shape, longitudinal shape, etc.), and/or transition time.

Any suitable operations may be performed when using pre-operative imaging to customize self-curling cochlear electrode leads for a recipient. For example, a first operation may include gathering the recipient's pre-operative images (e.g., CT, MRI, etc.). A second operation may include determining key landmarks and boundary outlines using image analysis software. A third operation may include computing a preferred trajectory (e.g., curvature, depth of insertion, etc.) of the self-curling cochlear electrode lead. A fifth operation may include designing the self-curling cochlear electrode lead including which type of shape memory polymer element(s) to use to achieve the preferred trajectory. A sixth operation may include fabricating and assembling the shape memory polymer element(s) within the self-curling cochlear electrode lead.

A self-curling cochlear electrode lead that includes a shape memory polymer element may be manufactured in any suitable manner. For example, a self-curling cochlear electrode lead may be formed in a molding process, an additive manufacturing process (e.g., a 3D printing process), a subtractive manufacturing process (e.g., etching, laser, etc.), and/or any other suitable manufacturing process. To illustrate an example, a first operation may include preparing a wire bundle and a protection tube. A second operation may include a welding block and loading the wire bundle. A third operation may include welding the wire bundle to a contact ring. A fourth operation may include creating a spine. A fifth operation may include creating a silicone carrier. A sixth operation may include combining the spine with the silicone carrier. A seventh operation may include creating rings. An eighth operation may include an array moulding operation. A ninth operation may include a shape memory polymer straightening and loading operation. A tenth operation may include molding the electrode tip, performing a final touch up, and/or adding a blue marker.

Shape memory polymer elements such as those described herein may be manufactured in any suitable manner. For example, a shape memory polymer formulation operation may include preparing a combination of several monomer solutions. A spin coating or casting process may be used to prepare sheets of shape memory polymer in a defined thickness. Shape memory polymer elements may then be cut from the sheets in any suitable manner. For example, laser cutting may be performed to cut shapes of shape memory polymer elements from the sheets.

In certain examples, a shape memory polymer element may be secured within a self-curling cochlear electrode lead to prevent the shape memory polymer element from shifting out of position during and/or after insertion into the cochlea. The shape memory polymer element may be secured in any suitable manner. For example, the shape memory polymer element may be secured by way of any suitable adhesive at a distal end and/or proximal end of the shape memory polymer element within the self-curling cochlear electrode lead. In certain alternative examples, the shape memory polymer element may be secured through a mechanical interlocking feature that may be molded into the self-curling cochlear electrode lead.

The shape memory polymer element may be embedded within a flexible body of a self-curling cochlear electrode lead. The flexible body of a self-curling cochlear electrode lead may be formed of any suitable biocompatible insulating material that is sufficiently flexible to bend and follow the lateral wall of the scala tympani and to further bend when the shape memory polymer element is subjected to a transition agent. In certain examples, the flexible body may be formed of silicone. However, any other suitable insulating material may be used in certain implementations. For example, materials such as polyimide, parylene, certain grades or blends of polyurethane and polyurethane/silicone materials, and/or any combination thereof may be used for the flexible body in certain implementations.

In certain examples, self-curling cochlear electrode leads such as those described herein may include one or more transition agent vias provided in the flexible body of the self-curling cochlear electrode leads. The transition agent vias in the flexible body are configured to facilitate controlling at least one an amount of curl or a rate of curl of a self-curling cochlear electrode lead. Such transition agent vias may provide a via (e.g., an opening) through which a transition agent such as, heat, water, and/or other bodily fluids may be transmitted into the flexible body to facilitate one or more shape memory polymer elements changing shape in a desired manner and/or at a desired rate.

Any suitable number and/or configuration of transition agent vias may be provided in a flexible body of a self-curling cochlear electrode lead as may serve a particular implementation. In certain examples, a plurality of transition agent vias may be provided along the length of a self-curling cochlear electrode lead. In such examples, the plurality of transition agent vias may be arranged in any suitable manner. For example, transition agent vias included in the plurality of transition agent vias may be spaced apart from one another evenly along a length of the flexible body in a longitudinal direction. Alternatively, at least some transition agent vias may be relatively closer to one another than other transition agent vias. For example, a first group of transition agent vias may be spaced apart from each other by a first distance and a second group of transition agent vias may be spaced apart from one another by a second distance that is different than the first distance. In certain alternative implementations, the plurality of transition agent vias may be provided only in a designated region of the self-curling cochlear electrode lead to increase the transition rate and/or degree of transition within that designated region.

In certain examples, the transition agent vias may correspond to hydrophilic transition agent vias that are configured to allow moisture to reach the shape memory polymer element(s). In certain examples, such hydrophilic transition agent vias may have any suitable surface coating that may render the transition agent vias hydrophilic. For example, the transition agent vias may be coated with any suitable higher surface energy material. Examples of such materials may include a hydrogel coating, certain metal coatings, and/or a zwitterionic coating. In certain examples, the transition agent vias may include modified silicone materials that are specifically configured to facilitate transport of either more or less moisture. The silicone materials may be modified in any suitable manner. For example, such modified silicone materials may be achieved by cross-linking or control of the free volume in the silicone. In certain examples, mobility of bonds in the silicone may also be used to increase moisture transport.

The transition agent vias may have any suitable size, shape, orientation, etc. as may serve a particular implementation. In certain examples, the transition agent vias may each have the same opening size and depth within a flexible body. In certain alternative examples, transition agent vias may have different opening sizes and/or depths into the flexible body. For example, the plurality of transition agent vias may include a first transition agent via having a first depth into the flexible body and a second transition agent via having a second depth into the flexible body, the first depth being greater than the second depth. Additionally or alternatively, the plurality of transition agent vias may in include a first transition agent via having a first width in a longitudinal direction of the flexible body and a second transition agent via having a second width in the longitudinal direction of the flexible body, the first width being greater than the second width. In such examples, the first width may be larger than the second width to facilitate relatively more liquid entering into the flexible body, which may cause the shape memory polymer element at the region of the first transition agent via to transition more quickly and/or to a greater degree as compared to other regions.

The transition agent vias in the flexible body may be configured in any suitable manner. In certain examples, the transition agent vias may include a plurality of cutouts in the flexible body. Such cutouts may facilitate entry of a transition agent (e.g., bodily fluid) toward a shape memory polymer element to trigger or otherwise facilitate transition of the shape memory polymer element. The configuration of such cutouts may be selected to control an amount of curl and/or a rate of curl of the self-curling cochlear electrode lead.

Transition agent vias in the form of cutouts may be formed in a flexible body of a self-curling cochlear implant lead in any suitable manner. For example, the cutouts may be formed by removing portions of the flexible body in any suitable manner after forming (e.g., molding) the flexible body. Alternatively, a mold used to form the flexible body may include a plurality of fins that are configured to form the cutouts when the flexible body is formed in the mold.

In certain examples, a transition agent via in a flexible body may be formed of a solid material instead of being a cutout. In such examples, the flexible body may have a first material property and the plurality of transition agent vias may have a second material property that is different than the first material property. The second material property may be configured to facilitate transmission of the transition agent through the flexible body and to the shape memory polymer element. To illustrate an example, the flexible body may be formed of silicone and the transition agent vias may be formed of a silicone composite that is configured to transmit bodily fluid and/or heat to the shape memory polymer element.

Transition agent vias may be provided along a length of a flexible body of a self-curling cochlear electrode lead in any suitable manner. For example, the flexible body may include a first portion that includes the plurality of transition agent vias and a second portion that does not include the plurality of transition agent vias. In certain examples, the first portion of the flexible body that includes the plurality of transition agent vias may be on a distal end of the self-curling cochlear electrode lead. In certain examples, the first portion of the flexible body that includes the plurality of transition agent vias is on a proximal end of the self-curling cochlear electrode lead. In certain examples, the first portion of the flexible body that includes the plurality of transition agent vias is on a medial portion of the self-curling cochlear electrode lead in relation to the plurality of electrode contacts.

The transition agent vias may be provided on any suitable side of a flexible body. In certain examples, the transition agent vias may be provided on a side of the flexible body that is opposite to the side of the flexible body with the plurality of electrode contacts.

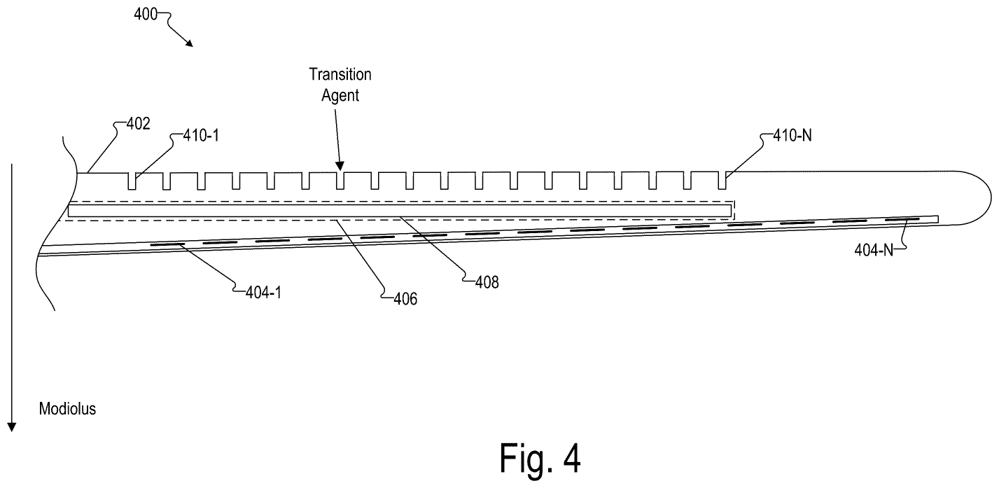

Illustrative configurations of self-curling cochlear electrode leads will now be described with respect to FIGS. 4-13.

FIG. 4 shows an exemplary self-curling cochlear electrode lead 400 that may be implemented in certain examples. As shown in FIG. 4, self-curling cochlear electrode lead 400 includes a flexible body 402 with a plurality of electrode contacts 404 (e.g., electrode contacts 404-1 through 404-N) arranged along a length of flexible body 402. Self-curling cochlear electrode lead 400 further includes a pre-formed lumen 406 with a shape memory polymer element 408 disposed therein. A plurality of transition agent vias 410 (e.g., transition agent vias 410-1 through 410-N) are also disposed along the length of flexible body 402 in a region corresponding to shape memory polymer element 408. Through transition agent vias 410, a transition agent (e.g., water, bodily fluid, etc.) may more easily enter into flexible body 402 to facilitate shape memory polymer element 408 transitioning to a curved shape during and/or after insertion into the cochlea. In the example shown in FIG. 4, transition agent vias 410 are evenly spaced apart from one another. However, it is understood that transition agent vias 410 may be spaced and/or positioned differently with respect to one another in certain alternative implementations. Transition agent vias 410 shown in FIG. 4 have square corners. However, it is understood that transition agent vias such as transition agent vias 410 may be configured differently in different examples. For example, transition agent vias 410 may have rounded bottom surfaces and/or rounded edges on an exterior surface of flexible body 402 in certain examples.

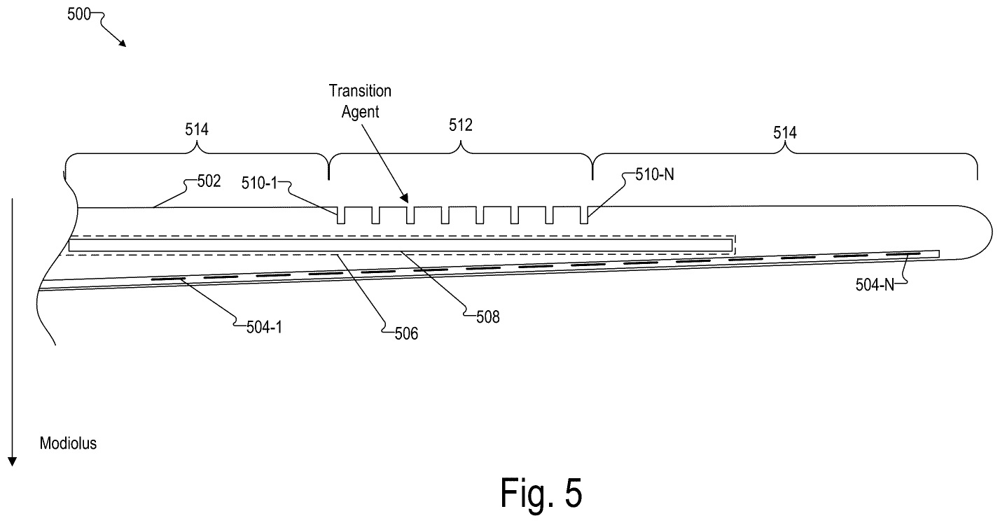

FIG. 5 shows another exemplary self-curling cochlear electrode lead 500 that may be implemented in certain examples. As shown in FIG. 5, self-curling cochlear electrode lead 500 includes a flexible body 502 with a plurality of electrode contacts 504 (e.g., electrode contacts 504-1 through 504-N) arranged along a length of flexible body 502. Self-curling cochlear electrode lead 500 further includes a pre-formed lumen 506 with a shape memory polymer element 508 disposed therein. A plurality of transition agent vias 510 (e.g., transition agent vias 510-1 through 510-N) are also disposed along the length of flexible body 502 in a region 512 corresponding to only a portion of shape memory polymer element 508. As shown in FIG. 5, regions 514 and 516 do not include any transition agent vias. Through transition agent vias 510, a transition agent (e.g., water, heat, bodily fluid, etc.) may more easily enter into flexible body 502 to facilitate shape memory polymer element 508 transitioning to a curved shape more quickly and/or to a greater extent in region 512 as compared to regions 514 and 516 of self-curling cochlear electrode lead 500.

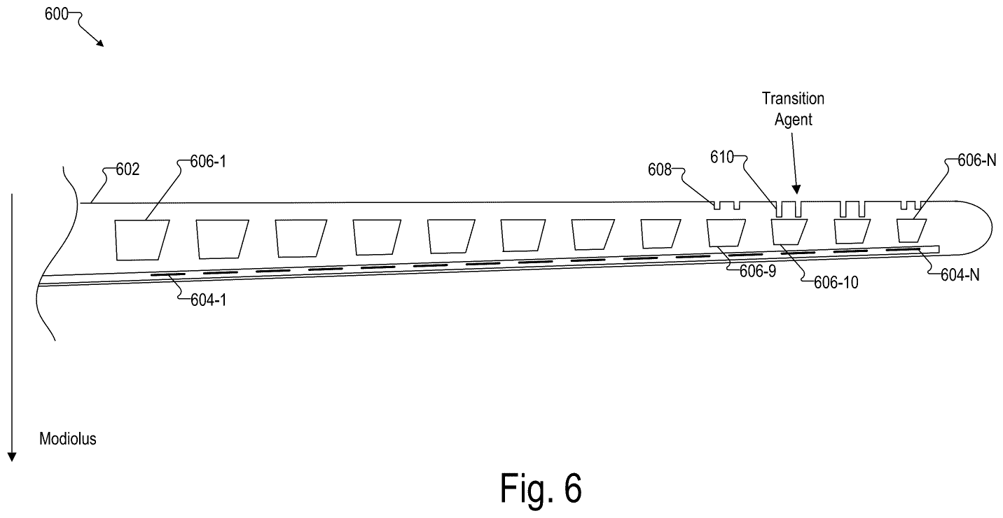

FIG. 6 shows another exemplary self-curling cochlear electrode lead 600 that may be implemented in certain examples. As shown in FIG. 6, self-curling cochlear electrode lead 600 includes a flexible body 602 with a plurality of electrode contacts 604 (e.g., electrode contacts 604-1 through 604-N) arranged along a length of flexible body 602. Self-curling cochlear electrode lead 600 further includes a plurality of shape memory polymer elements 606 (e.g., shape memory polymer elements 606-1 through 606-N) disposed along a length of flexible body 602. In the example shown in FIG. 6, transition agent vias having different depths are provided toward a distal end of self-curling cochlear electrode lead 600. For example, a transition agent via 608 has a first depth and a transition agent via 610 has a second depth that is greater than the first depth. Through transition agent vias 608 and 610 a transition agent (e.g., water, heat, bodily fluid, etc.) may more easily enter into flexible body 602 to facilitate shape memory polymer elements 606 on the distal end transitioning to a curved shape. In the example shown in FIG. 6, the relatively longer length of transition agent via 610 may result in shape memory polymer element 606-10 transitioning relatively more quickly than the adjacent shape memory polymer element 606-9.

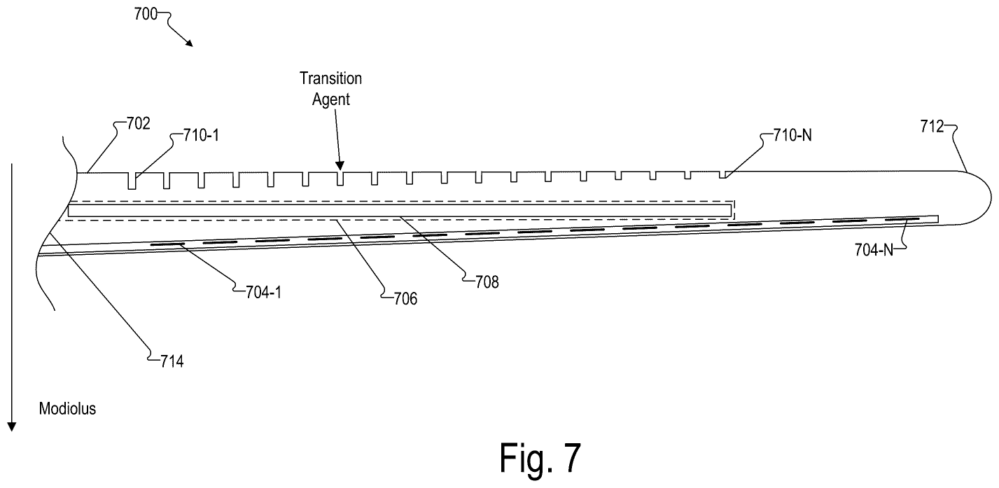

Because a distal end of a self-curling cochlear electrode lead is inserted first and, as a result, may be exposed longer to a transition agent, it may be desirable to configure transition agent vias such that a distal portion of a self-curling cochlear electrode lead curls relatively more slowly than a proximal portion of the self-curling cochlear electrode lead. FIG. 7 shows another exemplary self-curling cochlear electrode lead 700 that may be implemented in certain examples to facilitate different portions of the self-curling cochlear electrode lead curling at different rates. As shown in FIG. 7, self-curling cochlear electrode lead 700 includes a flexible body 702 with a plurality of electrode contacts 704 (e.g., electrode contacts 704-1 through 704-N) arranged along a length of flexible body 702. Self-curling cochlear electrode lead 700 further includes a pre-formed lumen 706 with a shape memory polymer element 708 disposed therein. A plurality of transition agent vias 710 (e.g., transition agent vias 710-1 through 710-N) are also disposed along the length of flexible body 702. In the example shown in FIG. 7, a depth of transition agent vias 710 increases from a distal end 712 to a proximal end 714 of self-curling cochlear electrode lead 700. Through transition agent vias 710, a transition agent (e.g., water, heat, bodily fluid, etc.) causes a distal portion of shape memory polymer element 708 to curl relatively more slowly a proximal portion of shape memory polymer element 708.

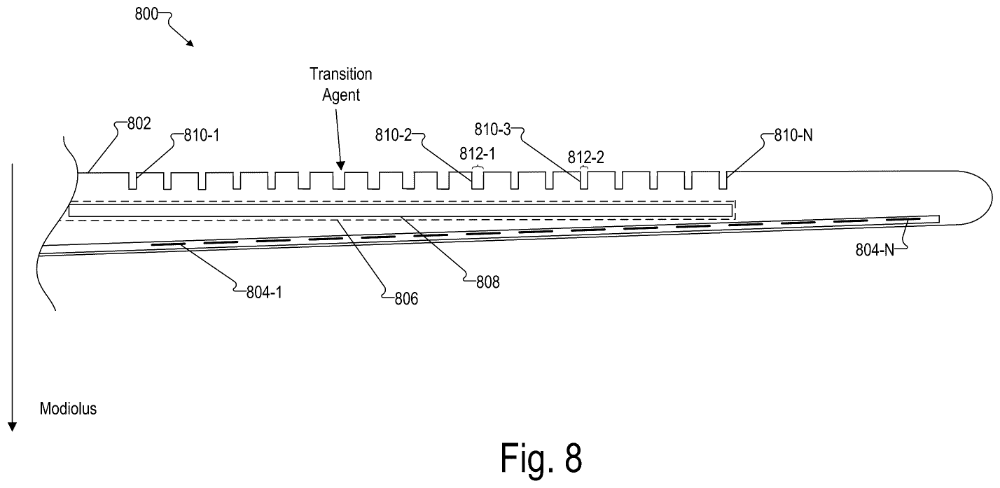

FIG. 8 shows another exemplary self-curling cochlear electrode lead 800 that may be implemented in certain examples to facilitate different curl rates in different regions of the self-curling cochlear electrode lead. As shown in FIG. 8, self-curling cochlear electrode lead 800 includes a flexible body 802 with a plurality of electrode contacts 804 (e.g., electrode contacts 804-1 through 804-N) arranged along a length of flexible body 802. Self-curling cochlear electrode lead 800 further includes a pre-formed lumen 806 with a shape memory polymer element 808 disposed therein. A plurality of transition agent vias 810 (e.g., transition agent vias 810-1 through 810-N) are also disposed along the length of flexible body 802. In the example shown in FIG. 8, a width of at least some of transition agent vias 810 is greater than other transition agent vias 810. For example, transition agent via 810-2 has a width of 812-1 in a longitudinal direction of self-curling cochlear electrode lead 800 and transition agent via 810-3 has a width of 812-2 that is relatively smaller than width 812-1 in a longitudinal direction. Through transition agent via 810-1, a middle portion of self-curling cochlear electrode lead 800 may curl at a relatively quicker rate and/or to a greater degree than the proximal and distal portions of self-curling cochlear electrode lead 800.

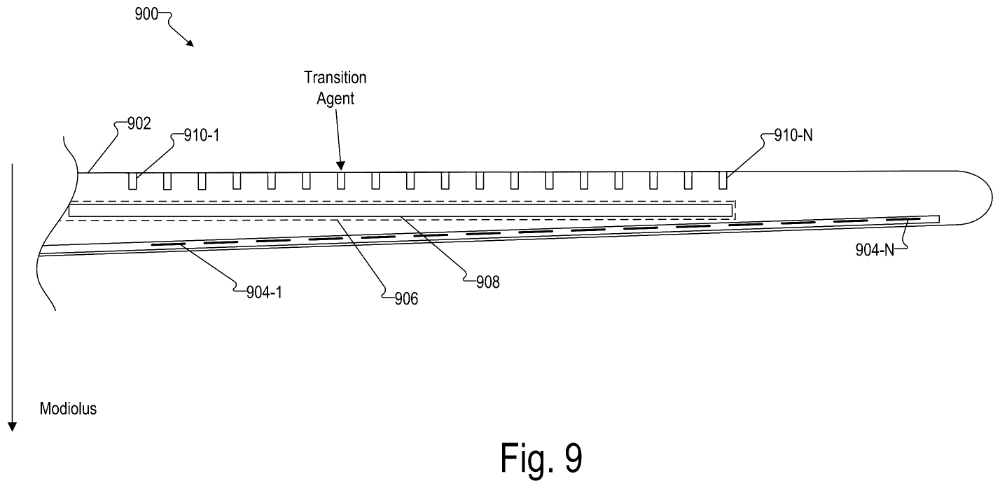

FIG. 9 shows another exemplary self-curling cochlear electrode lead 900 that may be implemented in certain examples. As shown in FIG. 9, self-curling cochlear electrode lead 900 includes a flexible body 902 with a plurality of electrode contacts 904 (e.g., electrode contacts 904-1 through 904-N) arranged along a length of flexible body 902. Self-curling cochlear electrode lead 900 further includes a pre-formed lumen 906 with a shape memory polymer element 908 disposed therein. A plurality of transition agent vias 910 (e.g., transition agent vias 910-1 through 910-N) are also disposed along the length of flexible body 902. In the example shown in FIG. 9, transition agent vias 910 include solid material as opposed to being cutouts. The material of transition agent vias is configured to facilitate transfer the transition agent to shape memory polymer element 908. In the example shown in FIG. 9, transition agent vias 910 may be formed of any suitable material as may serve a particular implementation. For example, transition agent vias 910 may be formed of a silicone composite that is configured to transmit the transition agent (e.g., heat, bodily fluid, etc.) to shape memory polymer element 908 to cause shape memory polymer element to curl.

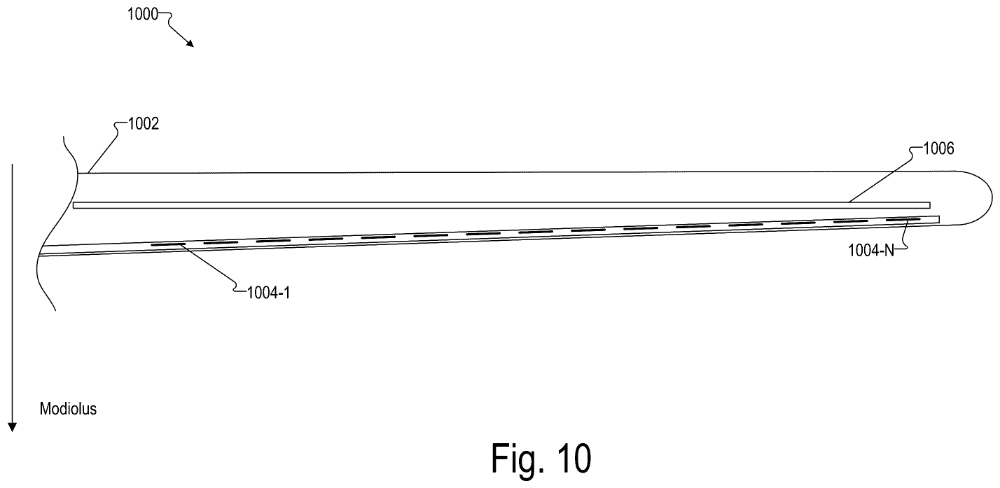

FIG. 10 shows another exemplary self-curling cochlear electrode lead 1000 that includes a flexible body 1002, a plurality of electrode contacts 1004 (e.g., electrode contacts 1004-1 through 1004-N, and a single shape memory polymer element 1006 that extends along a length of flexible body 1002. Although not shown in FIG. 10, it is understood that self-curling cochlear electrode lead 1000 may additionally include any suitable configuration and/or combination of transition agent vias such as described herein.

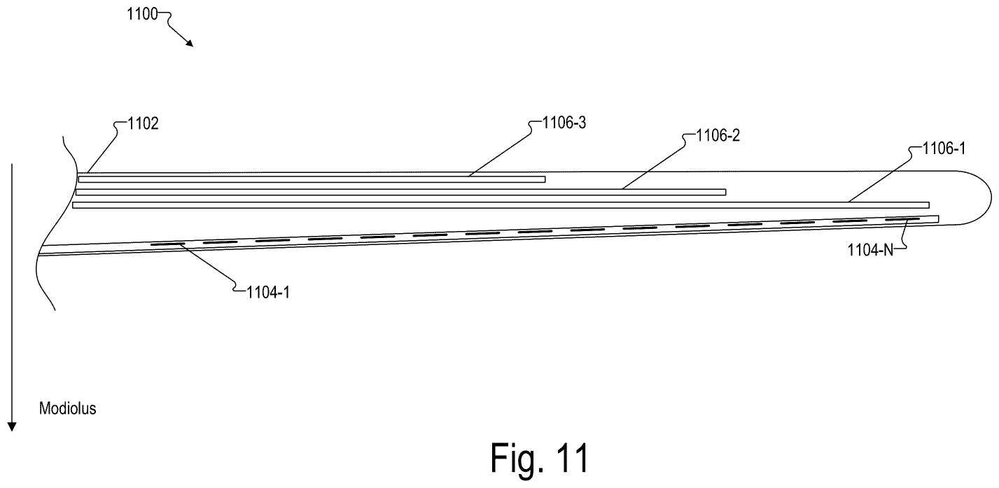

FIG. 11 shows another exemplary self-curling cochlear electrode lead 1100 that includes a flexible body 1102, a plurality of electrode contacts 1104 (e.g., electrode contacts 1104-1 through 1104-N, and a plurality of shape memory polymer elements 1106 (e.g., shape memory polymer elements 1106-1 through 1106-3) that extend along a length of flexible body 1102. As shown in FIG. 11, shape memory polymer elements 1106 have different lengths that may facilitate self-curling cochlear electrode lead 1100 curling during and/or after insertion into the cochlea. In the example shown in FIG. 11, the portion of self-curling cochlear electrode lead 1100 that includes each of shape memory polymer elements 1106-1, 1106-2, and 1106-3 may be relatively more stiff than and may curve more slowly than the portion of self-curling cochlear electrode lead 1100 that only includes shape memory polymer element 1106-1. Although not shown in FIG. 11, it is understood that self-curling cochlear electrode lead 1100 may additionally include any suitable configuration and/or combination of transition agent vias such as described herein.

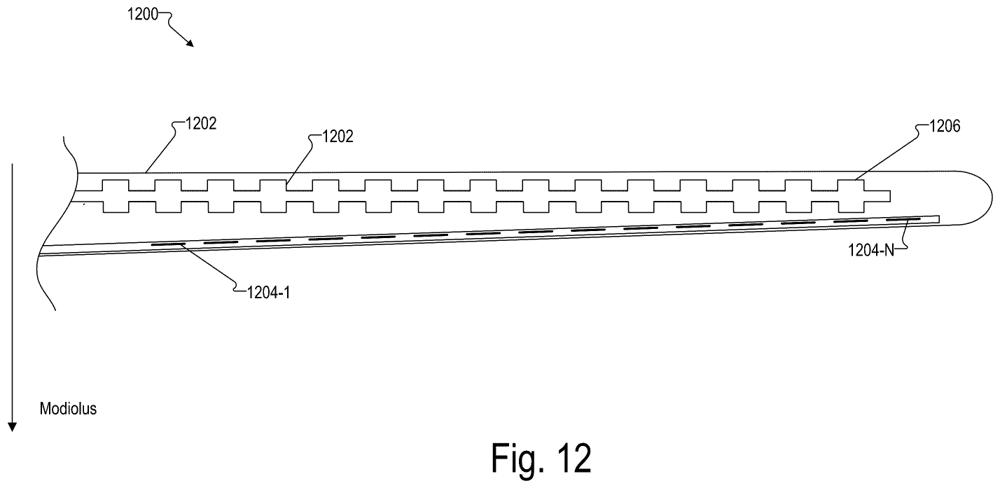

FIG. 12 shows another exemplary self-curling cochlear electrode lead 1200 that includes a flexible body 1202, a plurality of electrode contacts 1204 (e.g., electrode contacts 1204-1 through 1204-N), and a shape memory polymer element 1206 that extends along a length of flexible body 1202. In the example shown in FIG. 12, shape memory polymer element 1206 has a dog bone shape with a plurality of grooves 1208 along a longitudinal length of self-curling cochlear electrode lead 1200. Grooves 1208 shown in FIG. 12 are provided for illustrative purposes. It is understood that grooves 1208 may be configured differently in other implementations. For example, grooves 1208 may be wavelike with smooth as opposed to angular transitions in certain examples. Although not shown in FIG. 12, it is understood that self-curling cochlear electrode lead 1200 may additionally include any suitable configuration and/or combination of transition agent vias such as described herein.

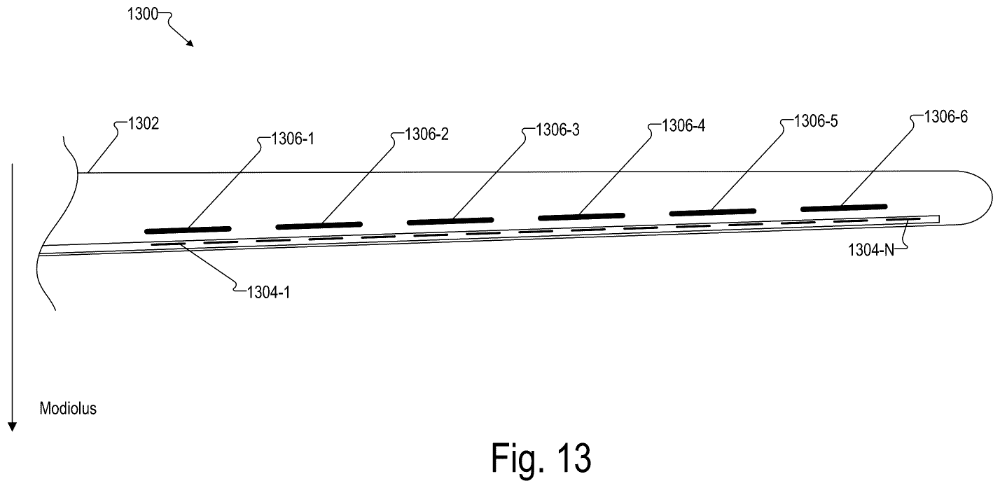

FIG. 13 shows another exemplary self-curling cochlear electrode lead 1300 that includes a flexible body 1302, a plurality of electrode contacts 1304 (e.g., electrode contacts 1304-1 through 1304-N), and a plurality of shape memory polymer elements 1306 (e.g., shape memory polymer elements 1306-1 through 1306-3) that extend along a length of flexible body 1302. In the example shown in FIG. 13, at least some of shape memory polymer elements 1306 may have a different transition rate and/or amount of curl as compared to at least some other shape memory polymer elements 1308. For example, shape memory polymer element 1306-6 may be configured to curl relatively more quickly and to a greater degree than shape memory polymer element 1306-1. Although not shown in FIG. 13, it is understood that self-curling cochlear electrode lead 1300 may additionally include any suitable configuration and/or combination of transition agent vias such as described herein.

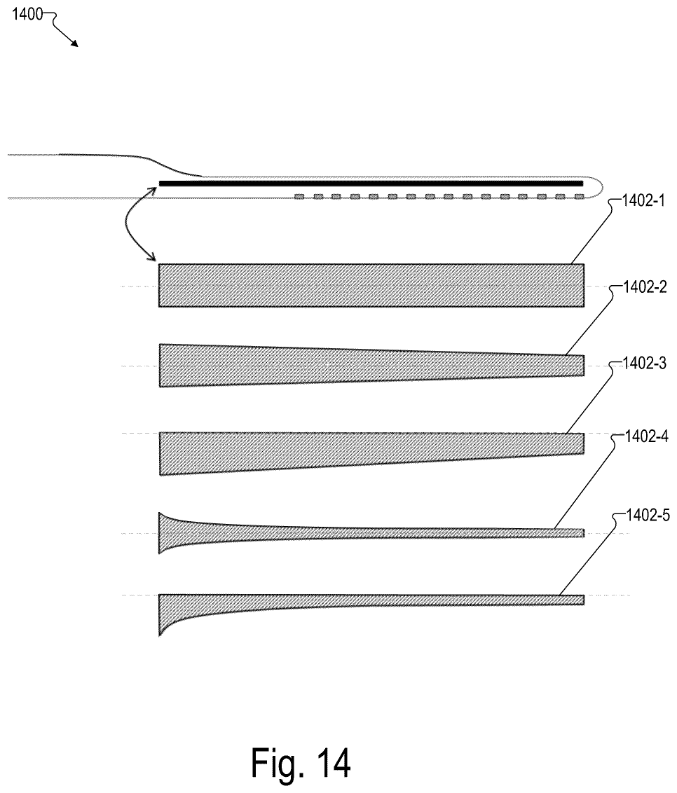

FIG. 14 shows a diagram 1400 depicting various different longitudinal shapes 1402 (e.g., shapes 1402-1 through 1402-5) that shape memory polymer elements may have according to principles described herein. For example, longitudinal shape 1402-1 has a rectangular shape, longitudinal shape 1402-4 has a “T” shape, and so forth. In certain examples, shape memory polymer elements may have a solid cross section. Alternatively, shape memory polymer elements may have a tube-shaped cross section.

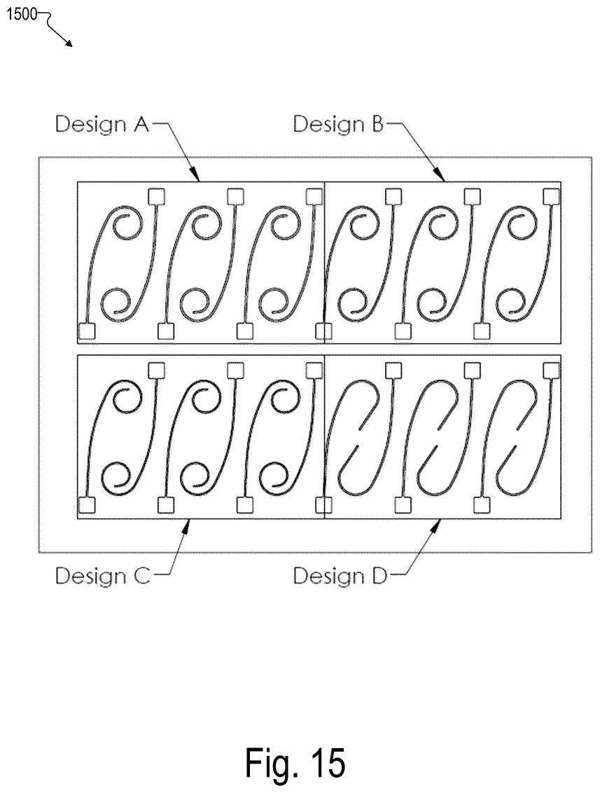

In certain examples, differently designed shape memory polymer elements may be cut from the shape sheet of shape memory polymer material. To illustrate, FIG. 15 shows an exemplary sheet 1500 of shape memory polymer material from which different shape memory polymer elements may be cut. As shown in FIG. 15, a Design A, a Design B, a Design C and a Design D may all be laser cut from the same sheet of shape memory polymer material.

Although self-curling cochlear electrode leads are described herein as including a shape memory polymer element, it is understood that a shape memory element made of any other suitable material other than a polymer may be used as may serve a particular implementation.

In certain examples, a computer-assisted surgical system may be implemented to facilitate inserting a self-curling cochlear electrode lead into a cochlea of a recipient. In such examples, the computer-assisted surgical system may include a robotic manipulating arm configured to control insertion of the self-curling cochlear electrode lead at a rate and trajectory that is optimal for the curling properties of the self-curling cochlear electrode lead. The robotic manipulating arm may be automatically controlled to insert the self-curling cochlear electrode lead along a pre-planned trajectory into the cochlea.

In certain examples, objective measurements may be used to facilitate curling of a self-curling cochlear electrode lead during insertion into the cochlea. In such examples, closed loop curling speed activation may be implemented based on objective measurements such as the distance that the self-curling cochlear electrode lead is from the modiolus/basilar membrane. Based on the distance, the curling speed may be changed or activated in any suitable manner. For example, based on the distance, a heating element within the self-curling cochlear electrode lead may activate to warm a shape memory polymer element and increase the curling speed of the shape memory polymer element. The distance from the modiolus/basilar membrane may be determined in any suitable manner. For example, a four point impedance measurement may be used in certain examples to determine the distance between the self-curling cochlear electrode lead and the modiolus/basilar membrane.

In certain examples, objective measurements may be used to determine the shape of a self-curling cochlear electrode lead and/or an amount of change of a shape memory polymer element in relation to the cochlea and whether to trigger further change of the shape memory polymer element. For example, impedance measurements such as monopolar, bipolar, three point or four point impedances may indicate how far a self-curling cochlear electrode lead is from a wall of the cochlea. In such examples, the objective measurements may be used to determine whether to activate another trigger of the shape memory polymer element. For example, moisture entering a flexible body of a self-curling electrode lead may cause a shape memory polymer element to curve a first amount. Based on the objective measurements, heat, light, and/or any other suitable input may be provided to trigger the shape memory to curve by a second amount that is in addition to the first amount.

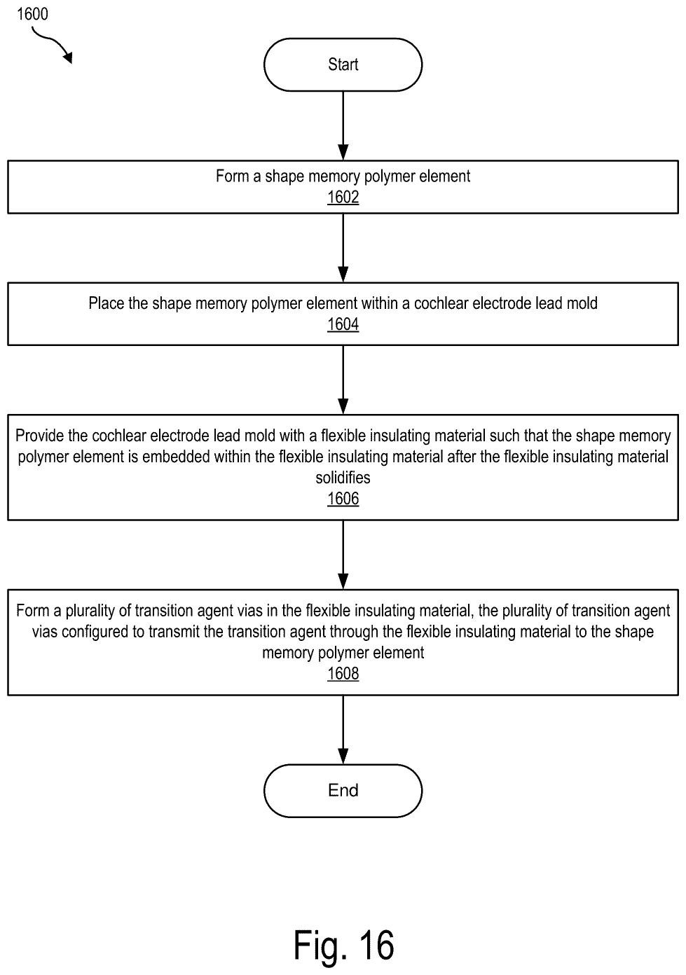

FIG. 16 illustrates a method 1600 for manufacturing a self-curling cochlear electrode lead (e.g., self-curling cochlear electrode lead 110). While FIG. 16 illustrates exemplary operations according to one embodiment, other embodiments may omit, add to, reorder, and/or modify any of the operations shown in FIG. 16.

In operation 1602, a shape memory polymer element is formed in any suitable manner. The shape memory polymer element is configured to cause the self-curling cochlear electrode lead to transition to a curved spiral shape so as to conform with a curvature of the human cochlea in response the shape memory polymer element being subjected to a transition agent. Operation 1602 may be performed in any of the ways described herein.

In operation 1604, the shape memory polymer element is placed in a cochlear electrode lead mold. The cochlear electrode lead mold may have any suitable configuration. In certain examples, the cochlear electrode lead mold may be a straight mold in which each of the plurality of electrode contacts are positioned in a straight line. Alternatively, the cochlear electrode lead mold may be a curved mold that has a curvature that conforms with the curvature of the human cochlea. Operation 1604 may be performed in any of the ways described herein.

In operation 1606, the cochlear electrode lead mold is provided with a flexible insulating material such that the shape memory polymer element is embedded within the flexible insulating material after the flexible insulating material solidifies. The cochlear electrode lead mold may be provided with the flexible insulating material in any suitable manner. In certain examples, the flexible insulating material may be injected into the cochlear electrode lead mold such that the flexible insulating material embeds the shape memory polymer element. Alternatively, the flexible insulating material may be compression molded in the cochlear electrode lead mold (e.g., by providing the flexible insulating material in a first half of the cochlear electrode lead mold and then pressing a second half of the cochlear electrode lead mold onto the flexible insulating material provided in the first half of the cochlear electrode lead mold). Operation 1606 may be performed in any of the ways described herein.

In operation 1608, a plurality of transition agent vias are formed in the flexible insulating material. The plurality of transition agent vias are configured to transmit a transition agent through the flexible insulating material to the shape memory polymer element. Operation 1608 may be performed in any of the ways described herein.

Advantages and features of the present disclosure can be further described by the following statements:

1. A self-curling cochlear electrode lead adapted for insertion into a human cochlea, comprising: a flexible body formed of a flexible insulating material; a shape memory polymer element that is embedded within the flexible body and that is configured to cause the self-curling cochlear electrode lead to transition to a curved spiral shape so as to conform with a curvature of the human cochlea in response the shape memory polymer element being subjected to a transition agent, wherein the flexible body includes a plurality of transition agent vias configured to transmit the transition agent through the flexible body to the shape memory polymer element.

2. The self-curling cochlear electrode lead of the preceding statement, wherein the transition agent includes a bodily fluid within a recipient of self-curling cochlear electrode lead.

3. The self-curling cochlear electrode lead of any of the preceding statements, wherein the transition agent includes heat applied to the self-curling cochlear electrode lead at least one of during insertion or after insertion.

4. The self-curling cochlear electrode lead of any of the preceding statements, wherein the heat is applied to the self-curling cochlear electrode lead by at least one of body temperature of a recipient of the self-curling cochlear electrode lead, an external heat source, or heat generated by way of the plurality of electrode contacts.

5. The self-curling cochlear electrode lead of any of the preceding statements, wherein: the flexible body includes a first portion that includes the plurality of transition agent vias and a second portion that does not include the plurality of transition agent vias.

6. The self-curling cochlear electrode lead of any of the preceding statements, wherein the first portion of the flexible body that includes the plurality of transition agent vias is on a distal end of the self-curling cochlear electrode lead.

7. The self-curling cochlear electrode lead of any of the preceding statements, wherein the first portion of the flexible body that includes the plurality of transition agent vias is on a proximal end of the self-curling cochlear electrode lead.

8. The self-curling cochlear electrode lead of any of the preceding statements, wherein the first portion of the flexible body that includes the plurality of transition agent vias is on a medial portion of the self-curling cochlear electrode lead in relation to the plurality of electrode contacts.

9. The self-curling cochlear electrode lead of any of the preceding statements, wherein the plurality of transition agent vias are configured to control at least one of an amount of curl or a rate of curl of the self-curling cochlear electrode lead.

10. The self-curling cochlear electrode lead of any of the preceding statements, wherein the plurality of transition agent vias are provided on an additional side of the flexible body that is opposite to the side of the flexible body with the plurality of electrode contacts.

11. The self-curling cochlear electrode lead of any of the preceding statements, wherein the plurality of transition agent vias includes a plurality of cutouts in the flexible body.

12. The self-curling cochlear electrode lead of any of the preceding statements, wherein the flexible body includes a hydrophilic coating in one or more cutout included in the plurality of cutouts.

13. The self-curling cochlear electrode lead of any of the preceding statements, wherein: the flexible body has a first material property and the plurality of transition agent vias have a second material property that is different than the first material property; and the second material property is configured to facilitate transmission of the transition agent through the flexible body and to the shape memory polymer element.

14. The self-curling cochlear electrode lead of any of the preceding statements, wherein the plurality of transition agent vias includes a first transition agent via having a first depth into the flexible body and a second transition agent via having a second depth into the flexible body, the first depth being greater than the second depth.

15. The self-curling cochlear electrode lead of any of the preceding statements, wherein the plurality of transition agent vias includes a first transition agent via having a first width in a longitudinal direction of the flexible body and a second transition agent via having a second width in the longitudinal direction of the flexible body, the first width being greater than the second width.

16. The self-curling cochlear electrode lead of any of the preceding statements, wherein transition agent vias included in the plurality of transition agent vias are spaced apart from one another evenly along a length of the flexible body in a longitudinal direction.

17. A self-curling cochlear electrode lead adapted for insertion into a human cochlea, comprising: a flexible body formed of a flexible insulating material; a plurality of shape memory polymer elements embedded within the flexible body, each shape memory polymer element included in the plurality of shape memory polymer elements configured to cause the self-curling cochlear electrode lead to transition to a curved spiral shape so as to conform with a curvature of the human cochlea in response the plurality of shape memory polymer elements being subjected to a transition agent, wherein the flexible body includes a plurality of transition agent vias configured to transmit the transition agent through the flexible body a subset of shape memory polymer elements included in to the plurality of shape memory polymer elements.

18. A method of manufacturing a self-curling cochlear electrode lead adapted for insertion into a human cochlea, the method comprising: forming a shape memory polymer element that is configured to cause the self-curling cochlear electrode lead to transition to a curved spiral shape so as to conform with a curvature of the human cochlea in response the shape memory polymer element being subjected to a transition agent; placing the shape memory polymer element within a cochlear electrode lead mold; providing, after placing the shape memory polymer element within the cochlear electrode lead mold, the cochlear electrode lead mold with a flexible insulating material such that the shape memory polymer element is embedded within the flexible insulating material after the flexible insulating material solidifies; and forming a plurality of transition agent vias in the flexible insulating material, the plurality of transition agent vias configured to transmit the transition agent through the flexible insulating material to the shape memory polymer element.

19. The method of the preceding statement, wherein the forming of the plurality of transition agent vias includes forming a plurality of cutouts in the flexible insulating material.

20. The method of any of the preceding statements, further comprising providing a hydrophilic coating in one or more cutouts included in the plurality of cutouts.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A self-curling cochlear electrode lead adapted for insertion into a human cochlea, comprising:
a flexible body formed of a flexible insulating material;
a shape memory polymer element that is embedded within the flexible body and that is configured to cause the self-curling cochlear electrode lead to transition to a curved spiral shape so as to conform with a curvature of the human cochlea in response the shape memory polymer element being subjected to a transition agent,
wherein the flexible body includes a plurality of transition agent vias configured to transmit the transition agent through the flexible body to the shape memory polymer element.

2. The self-curling cochlear electrode lead of claim 1, wherein the transition agent includes a bodily fluid within a recipient of self-curling cochlear electrode lead.

3. The self-curling cochlear electrode lead of claim 1, wherein the transition agent includes heat applied to the self-curling cochlear electrode lead at least one of during insertion or after insertion.

4. The self-curling cochlear electrode lead of claim 3, wherein the heat is applied to the self-curling cochlear electrode lead by at least one of body temperature of a recipient of the self-curling cochlear electrode lead, an external heat source, or heat generated by way of the plurality of electrode contacts.

5. The self-curling cochlear electrode lead of claim 1, wherein:
the flexible body includes a first portion that includes the plurality of transition agent vias and a second portion that does not include the plurality of transition agent vias.

6. The self-curling cochlear electrode lead of claim 5, wherein the first portion of the flexible body that includes the plurality of transition agent vias is on a distal end of the self-curling cochlear electrode lead.

7. The self-curling cochlear electrode lead of claim 5, wherein the first portion of the flexible body that includes the plurality of transition agent vias is on a proximal end of the self-curling cochlear electrode lead.

8. The self-curling cochlear electrode lead of claim 5, wherein the first portion of the flexible body that includes the plurality of transition agent vias is on a medial portion of the self-curling cochlear electrode lead in relation to the plurality of electrode contacts.

9. The self-curling cochlear electrode lead of claim 1, wherein the plurality of transition agent vias are configured to control at least one of an amount of curl or a rate of curl of the self-curling cochlear electrode lead.

10. The self-curling cochlear electrode lead of claim 1, wherein the plurality of transition agent vias are provided on an additional side of the flexible body that is opposite to the side of the flexible body with the plurality of electrode contacts.

11. The self-curling cochlear electrode lead of claim 1, wherein the plurality of transition agent vias includes a plurality of cutouts in the flexible body.

12. The self-curling cochlear electrode lead of claim 11, wherein the flexible body includes a hydrophilic coating in one or more cutout included in the plurality of cutouts.

13. The self-curling cochlear electrode lead of claim 1, wherein:
the flexible body has a first material property and the plurality of transition agent vias have a second material property that is different than the first material property; and
the second material property is configured to facilitate transmission of the transition agent through the flexible body and to the shape memory polymer element.

14. The self-curling cochlear electrode lead of claim 1, wherein the plurality of transition agent vias includes a first transition agent via having a first depth into the flexible body and a second transition agent via having a second depth into the flexible body, the first depth being greater than the second depth.

15. The self-curling cochlear electrode lead of claim 1, wherein the plurality of transition agent vias includes a first transition agent via having a first width in a longitudinal direction of the flexible body and a second transition agent via having a second width in the longitudinal direction of the flexible body, the first width being greater than the second width.

16. The self-curling cochlear electrode lead of claim 1, wherein transition agent vias included in the plurality of transition agent vias are spaced apart from one another evenly along a length of the flexible body in a longitudinal direction.

17. A self-curling cochlear electrode lead adapted for insertion into a human cochlea, comprising:
a flexible body formed of a flexible insulating material;
a plurality of shape memory polymer elements embedded within the flexible body, each shape memory polymer element included in the plurality of shape memory polymer elements configured to cause the self-curling cochlear electrode lead to transition to a curved spiral shape so as to conform with a curvature of the human cochlea in response the plurality of shape memory polymer elements being subjected to a transition agent,
wherein the flexible body includes a plurality of transition agent vias configured to transmit the transition agent through the flexible body a subset of shape memory polymer elements included in to the plurality of shape memory polymer elements.

18. A method of manufacturing a self-curling cochlear electrode lead adapted for insertion into a human cochlea, the method comprising:
forming a shape memory polymer element that is configured to cause the self-curling cochlear electrode lead to transition to a curved spiral shape so as to conform with a curvature of the human cochlea in response the shape memory polymer element being subjected to a transition agent;
placing the shape memory polymer element within a cochlear electrode lead mold;
providing, after placing the shape memory polymer element within the cochlear electrode lead mold, the cochlear electrode lead mold with a flexible insulating material such that the shape memory polymer element is embedded within the flexible insulating material after the flexible insulating material solidifies; and
forming a plurality of transition agent vias in the flexible insulating material, the plurality of transition agent vias configured to transmit the transition agent through the flexible insulating material to the shape memory polymer element.

19. The method of claim 18, wherein the forming of the plurality of transition agent vias includes forming a plurality of cutouts in the flexible insulating material.

20. The method of claim 19, further comprising providing a hydrophilic coating in one or more cutouts included in the plurality of cutouts.
